(19) United States
(12) Patent Application Publication
(10) Pub. No.: US 2010/0092995 A1
(43) Pub. Date: Apr. 15, 2010

(54) FLUORESCENT RUTHENIUM COMPOUNDS FOR DETECTING CALCIUM BINDING PROTEINS

(76) Inventors: Varda Shoshan-Barmatz, Omer (IL); Adrian Israelson, Beer Sheva (IL); Daniela Lemelson, Sderot (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(21) Appl. No.: 12/443,850

(22) PCT Filed: Oct. 7, 2007
(86) PCT No.: PCT/IL2007/001210

§ 371 Date: Apr. 14, 2009

(30) Foreign Application Priority Data
Oct. 5, 2006 (IL) 178494

(51) Int. Cl. G01N 33/53; C07D 311/02

(52) U.S. Cl. 435/7.1; 549/212; 534/10

(57) ABSTRACT

A fluorescent compound exhibiting a high affinity to calcium-binding proteins (CaBP) is provided. The compound, containing ruthenium, enables to detect, identify, and isolate CaBPs involved in cellular signaling and regulation. The compound is employed for diagnosing a disorder associated with CaBPs defects.

### FIELD OF THE INVENTION

The present invention relates to fluorescent ruthenium probes that bind to calcium-binding proteins, providing a new tool for characterizing and localizing those proteins by detecting fluorescence, optionally further employing radioactive ¹⁰³Ru.

### BACKGROUND OF THE INVENTION

Calcium (Ca²⁺) is a ubiquitous intracellular signal transducer, responsible for controlling numerous cellular processes. It regulates muscle contraction, neurotransmitter release, hormone secretion, cell motility, mitosis, and gene expression. Ca²⁺-signaling is employed throughout the life of the organism, beginning with a surge of Ca²⁺ at fertilization and ending with the induction of apoptosis at cell death.

Several dozens of Ca²⁺-binding proteins (CaBP), having high-affinity or low-affinity Ca²⁺-binding sites, have been identified, purified and characterized, and their functions were established. However, as judged by the significant amount of Ca²⁺-dependent activities without known participants—many CaBP are yet unidentified. For example, mitochondria contain several different systems for Ca²⁺ transport which are waiting for elucidation. An important aspect in untangling the complex web of signals, receptors, and pathways is the precise localization of the CaBP in the organism and tracking their intracellular movements.

Several reagents interacting with Ca²⁺-binding proteins were used in various studies, among them ruthenium red (RuR) and Ru360. WO 2005/079155, of the instant inventors, disclosed a ruthenium compound (AzRu) specifically binding to Ca²⁺-binding proteins and covalently linking to them after photoactivation. AzRu was shown to inhibit some processes which were not affected by RuR or Ru360, such as the SR Ca²⁺-pump and others. A recent study, explaining the mechanism of inhibiting a potassium channel by RuR [Czirjak G. et al.: Mol. Pharmacol. 63 (2003) 646-52] indicated how important the charge and its location for the activity of a reagent is. A strong need is therefore felt for new reagents of different activities and specificities, particularly reagents enabling to track and locate CaBP within their environment. It is therefore an object of this invention to provide a compound binding specifically to calcium-binding proteins and visualize them in situ.

It is another object of this invention to provide a fluorescent probe that specifically binds to Ca²⁺-binding sites of calcium-binding proteins and labels them.

It is still another object of this invention to provide a ruthenium-based probe which specifically binds to Ca²⁺-binding sites of calcium-binding proteins, thereby allowing to localize and characterize such sites.

It is further an object of this invention to provide diagnostic uses of Ru-based fluorescent compounds for labeling proteins associated with defects in the function of a Ca²⁺-related signal pathways.

Other objects and advantages of the present invention will appear as the description proceeds.

### SUMMARY OF THE INVENTION

The invention provides fluorescent, ruthenium-containing, compounds (FlRu) which bind to a calcium-binding protein. A compound of the invention binds to different Ca²⁺-binding proteins with different affinities. Different compounds of the invention bind to a certain protein with different affinities, providing a range of compounds for protein characterization. A compound according to the invention may be characterized by its binding to an array of standard proteins, among them being, for example, troponin. In a preferred embodiment of the invention, the fluorescent compound binds to troponin, wherein the binding affinity is characterized by an association constant of at least about 10⁵. The fluorescent compound of the invention containing ruthenium (FlRu) may comprise, beside ruthenium, a fluorescent moiety selected, for example, from rhodamine or tetramethyl rhodamine moiety, or cyanine-based moiety. Said moiety and ruthenium are usually present in a molar ratio 1:2 in the complex. The compound of the invention is obtained by a reaction between an azido-ruthenium compound and a fluorescent reagent, wherein said azido-ruthenium compound is preferably the compound described in WO 2005/079155 (AzRu). The term fluorescent reagent relates to a compound, or a mixture of compounds, usually isomers, which contain in their molecules a strong fluorophore (fluorescent moiety), and which are capable to covalently bind a fluorescent moiety to macromolecular targets, such as proteins, under relatively mild conditions. An example of such activated fluorescent agent, without wishing to be limited to them, is FITC, EITC, RITC, TITC, and Cy5. The compound of the invention may label a target molecule in more ways, beside rendering the target fluorescent, the compound may comprise a radioactive isotope. In a preferred embodiment, the AzRu containing ¹⁰³Ru is employed. The compound of the invention binds usually non-covalently to CaBP, thereby labeling the proteins by fluorescent tag. The combination of a fluorescent moiety, a linkage group, and a CaBP decides how strong said noncovalent interaction will be, under certain condition comprising ionic strength, pH, temperature, etc., and a user of the invention will choose the parameters in accordance with her/his experimental needs. In a preferred embodiment of the invention, a fluorescent, Ru-based, compound binds to a calcium binding site of said calcium-binding protein. Said compound binds non-covalently to a calcium-binding protein, thereby labeling a Ca²⁺-binding site of said protein by a fluorescent tag. In one aspect of the invention, FlRu specifically bind to CaBPs, thereby inhibiting their calcium-binding activity. Said calcium-binding proteins may belong to proteins involved in signal transduction, muscle contraction, neurotransmitter release, hormone secretion, cell motility, apoptosis, fertilization, cell proliferation, cell mitosis and in gene expression; proteins associated with Ca²⁺-transport, Ca²⁺-pumps, and with the mitochondrial uniporter; channel protein VDAC; Ca²⁺-release channel/ryanodine receptor; IP₃ receptor proteins involved in Ca²⁺-efflux in mitochondria; and soluble Ca²⁺ binding proteins regulating various cellular activities.

The invention relates to a method of detecting a calcium-binding protein source containing CaBP, comprising i) providing a source containing a CaBP; ii) contacting said source with a FlRu compound as described above, whereby said CaBP is bound to said compound; and iii) removing unbound FlRu; thereby identifying CaBP as a fluorescent material that remains after the removal of the low-molecular FlRu in step iii). Said source in the method of the invention may comprise a living cell, in which FlRu may interact with cellular proteins, labeling some of them. Said source may comprise an in vitro sample, containing either soluble proteins or proteins embedded in membranes, wherein some proteins bind more and some less of the fluorescent compound, and/or wherein more compounds may be applied simultaneously or in separate samples to distinguish between different groups of proteins. Said source may comprise, for example, an array of microsamples or a protein chip.

The invention is directed to a method of isolating a calcium-binding protein from a source comprising the same, which method comprises the steps of: i) providing a source containing a CaBP; ii) contacting said source with a FlRu described above, whereby said CaBP is bound to said compound; iii) removing the unbound FlRu; and iv) isolating fluorescent material that remains after the removal of the unbound low-molecular FlRu in step iii). Said method preferably further comprises characterizing the structure of said CaBP by utilizing method selected from the group consisting of electrophoresis, autoradiography, liquid chromatography, MALDI-TOF analysis, LC-MS/MS, protein sequencing and a sequence homology search. The invention is also directed to a method of screening for calcium-binding proteins, comprising the steps of: i) providing a test sample comprising proteins; ii) contacting said sample with a FlRu compound under conditions which allow noncovalent binding of said compound to calcium-binding proteins to form fluorescent protein complexes; iii) isolating from said sample said fluorescent complexes; and optionally iv) subjecting said complexes to conditions supporting the release of said compound from said complexes, thereby obtaining free calcium-binding proteins. Said method of screening preferably further comprises the step of testing the proteins obtained in step iv) for their calcium binding activity. In an important aspect, the methods provided in the invention, advantageously, utilize FlRu comprising ¹⁰³Ru.

The invention provides a process for preparing a fluorescent ruthenium-based containing compound which binds to a calcium-binding protein, comprising: i) providing AzRu; ii) contacting AzRu with a reactive fluorescent compound in a solvent; iii) removing unreacted AzRu and unreacted activated compound, thereby obtaining a FlRu compound; and optionally iv) removing solvents from said compound obtained in step iii). Said removing preferably comprises a chromatographic method, wherein, e.g., a gel filtration, or other column may be used. Said fluorescent reagent may comprise FITC, EITC, RITC, TITC, Cy5, or other known compounds containing a strong fluorophore. In a preferred embodiment, said activated fluorescent compound is FITC or EITC. Said FlRu is, in a preferred embodiment, complex obtained by reacting FITC or EITC with AzRu. For example, 10 mM FITC and 10 mM AzRu in 50% DMF are incubated overnight at room temperature. The product, denoted as FITC-Ru, has Rf of about 0.6 at TLC on silica gel F₂₅₄ plates, using a developer consisting of 80% of 1M ammonium formate and 20% methanol, pH 8.5. Said complex (FITC-Ru) is well soluble in DMF and DMSO, and less soluble in water, ethanol and methanol. The absorbance maximum of the complex is at about 495 nm, and a fluorescent emission maximum at 525 nm when excited by 495 nm light.

In another embodiment of the preparation method of the invention, 8 mM EITC is contacted with 7 mM AzRu in 68% DMF, and incubated overnight at room temperature. Resulting EITC-Ru complex has Rf of about 0.7 at TLC on silica gel F₂₅₄ plates, using a developer containing ethyl acetate 60%, methanol 30%, and water 10%. Said complex is soluble in DMF, methanol, and DMSO, less soluble in water, and insoluble in ethanol, n-butanol and diethyl-ether. Said EITC-Ru has an absorbance maximum at about 528 nm, an excitation maximum at 518 nm, and a fluorescent emission maximum at 539 nm.

The invention further aims at a method of diagnosing a disorder associated with a defect in the function of a CaBP in a subject, comprising: i) providing a source of a CaBP of said subject, and a control source of a CaBP of a normal subject; ii) contacting said source with said compound under conditions suitable for binding to occur; and iii) detecting the fluorescence in the source. Said method may comprise providing said source, contacting them with said compound, optionally processing said sources to enrich them with said CaBP, detecting the fluorescence patterns in the samples, and comparing said pattern for said subject with the pattern obtained for said control sample. Said source may comprise cells, enriched protein samples, and protein chips.

### BRIEF DESCRIPTION OF THE DRAWINGS

The above and other characteristics and advantages of the invention will be more readily apparent through the following examples, and with reference to the appended drawings, wherein:

FIG. 1 shows the purification of FITC-Ru and FITC-¹⁰³Ru on Sephadex LH-20 column;

FIG. 2 shows the characterization of FITC-Ru on by TLC;

FIG. 3 shows UV spectra of FITC-Ru, FIG. 3A being an absorbance spectrum, and FIG. 3B being an emission spectrum with excitation of 495 nm;

FIG. 4 demonstrates the inhibition by FITC-Ru of the Ca²⁺-accumulation, FIG. 4A relates to SR membranes, FIG. 4B relates to isolated mitochondria;

FIG. 5 shows the structural formula of FITC-Ru;

FIG. 6 shows the excitation (FIG. 6A) and emission (FIG. 6B) spectra of EITC-Ru;

FIG. 7 shows the inhibition by EITC-Ru, FITC-Ru and by AzRu of the Ca²⁺-accumulation in SR membranes;

FIG. 8 shows the inhibition by EITC-Ru of the Ca²⁺-accumulation in isolated mitochondria;

FIG. 9 shows the effects of FITC-Ru, EITC-Ru and AzRu on ryanodine-binding by SR membranes; and

FIG. 10 demonstrates fluorescence of FITC-Ru, of a complex FITC-Ru with troponin, and of a purified troponin at excitation by 495 nm, FIG. 10A shows the emission spectra, FIG. 10B shows the dependence of ΔF for FITC-Ru and the complex FITC-Ru-troponin on the FITC-Ru concentration.

### DETAILED DESCRIPTION OF THE INVENTION

The Ru-based reagent, AzRu, described in WO 2005/079155 [see also Israelson A. et al.: Chem. Biol. 12 (2005) 1169-78] provides an invaluable means to reach Ca²⁺-binding proteins (CaBP), to label them, to covalently bind them, or to catch them, enabling also radio-detection when using ¹⁰³Ru isotope. It has now been found that some of the advantageous properties of AzRu may be employed in a new probe that further enables fluorescent labeling of the target CaBP. The new fluorescent probes were obtained by reacting AzRu with FITC (fluorescein isothiocyanate) or EITC (eosin isothiocyanate). The new compounds, denoted FITC-Ru and EITC-Ru, were found to inhibit Ca²⁺-uptake by SR also accumulation of Ca²⁺ in mitochondria. Further it was found that FITC-Ru interacts with the purified troponin, and the fluorescence of the compound was quenched upon its binding to troponin. Thus, a new reagent obtained, specifically interacting with Ca²⁺-binding proteins and labeling biological structures containing them.

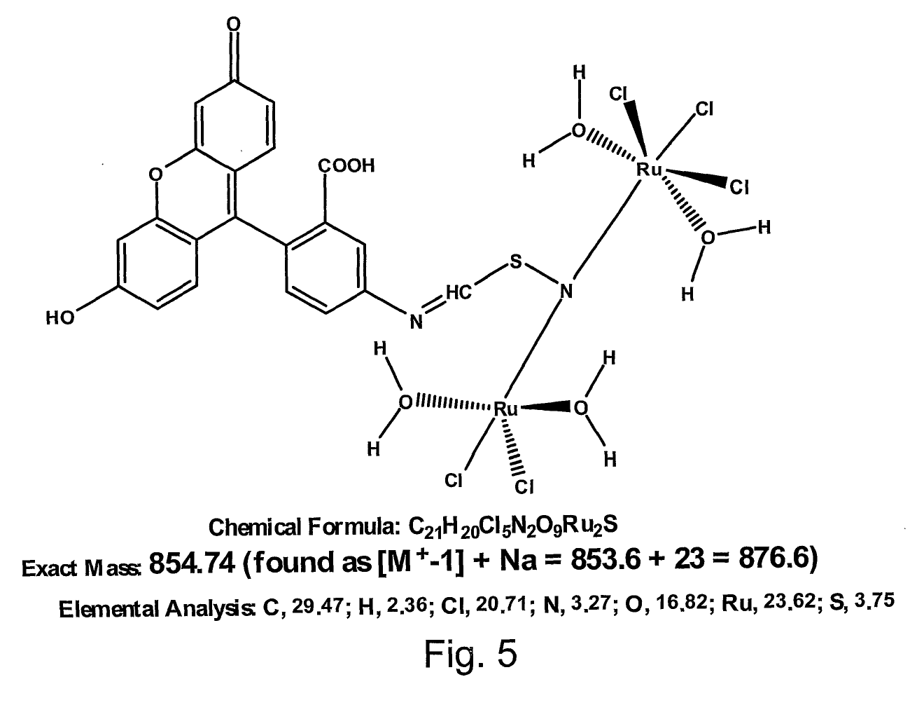

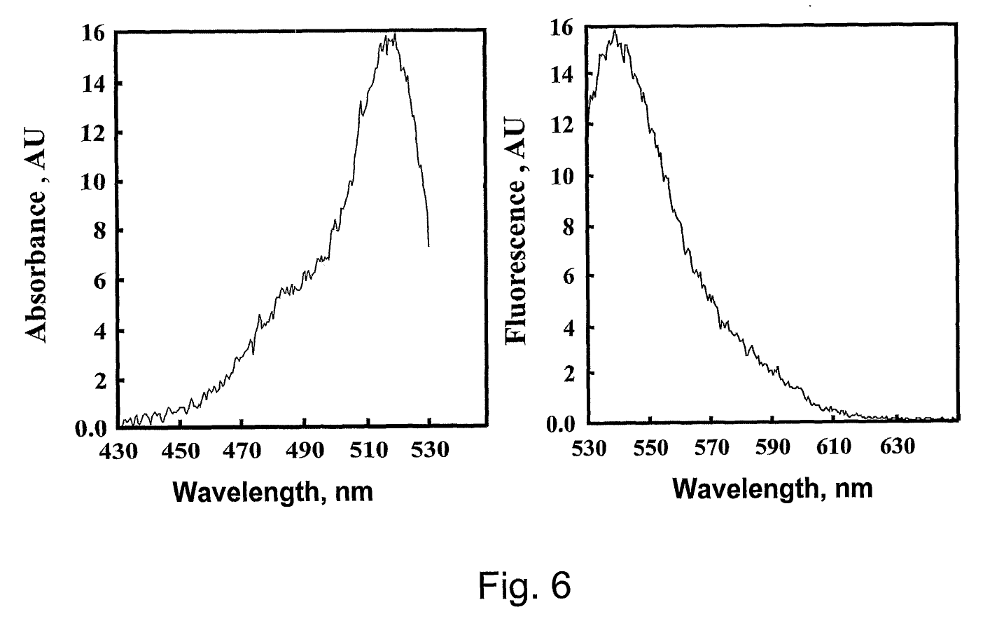

The above finding provides a new, useful, tool for characterizing the CaBP, enabling fluorescent labeling of structures comprising CaBP, and furthermore, due to the presence of ruthenium in the reagent, enabling a second type of labeling effected by one molecule—radiolabeling by ¹⁰³Ru. The multi-mode labeling reagent may be obtained, for example, by a reaction of FITC with [¹⁰³Ru]AzRu, followed by chromatographic purification. The reagent is soluble in water and DMSO and its structure was determined using LC/MS/MS (FIG. 5). The preparation of FITC-Ru comprising radioactive ¹⁰³Ru enabled to estimate its molar concentration based on the known specific radioactivity of Ru. The molar extinction coefficient was thus estimated to be about 92,000 in water.

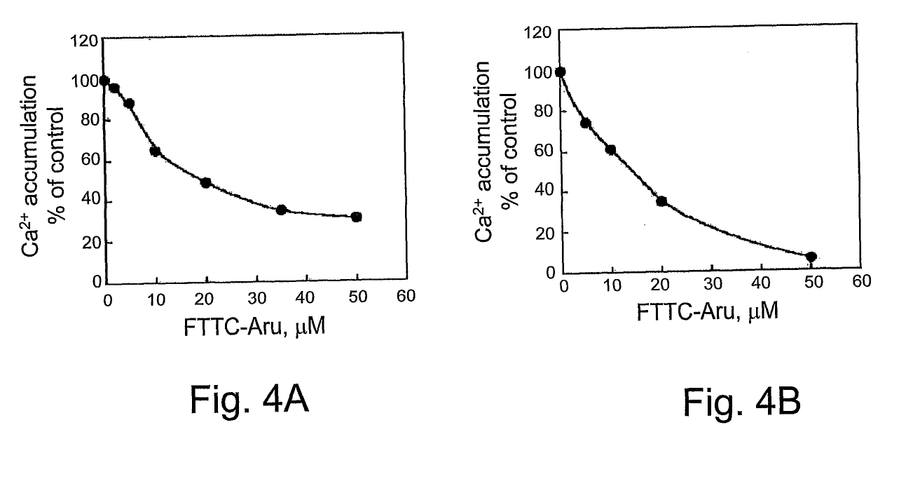

The capability of the reagent to inhibit Ca²⁺-dependent processes was examined in various in vitro systems. It was found that FITC-Ru inhibits Ca²⁺ accumulation by SR (FIG. 4A), with C₅₀ (the concentration providing 50% of the maximal inhibition) being about 15 μM. The reagent at a concentration of 50 μM caused 80% to 90% inhibition. Similar results were obtained also with inhibiting the Ca²⁺-uptake in mitochondria (FIG. 4B).

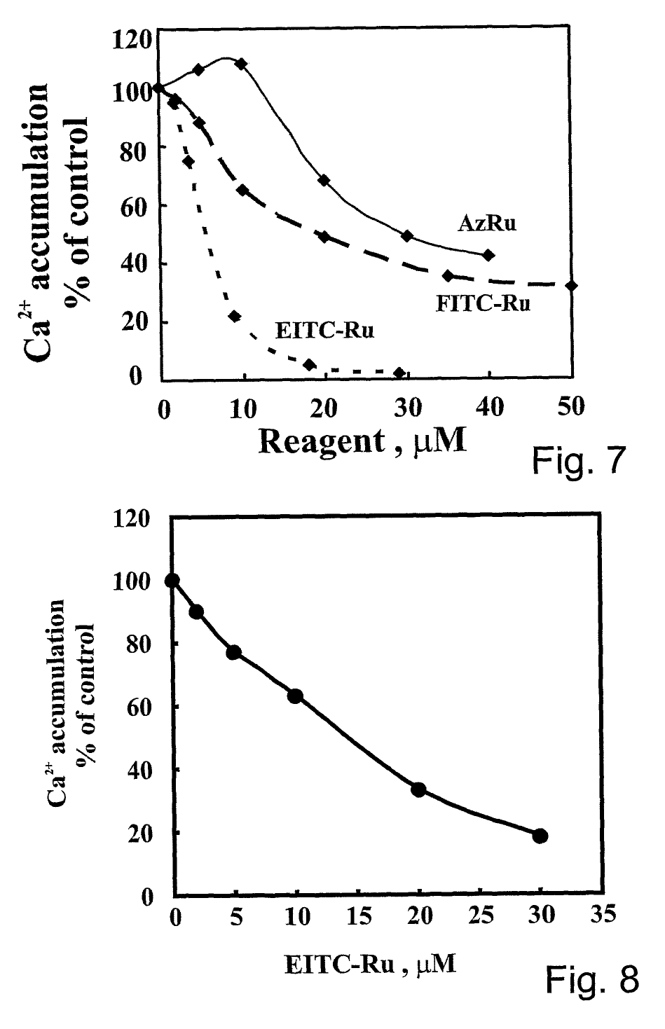

EITC-Ru was found to inhibit Ca²⁺ accumulation by SR membranes (FIG. 7), with C₅₀ being about 7 μM.

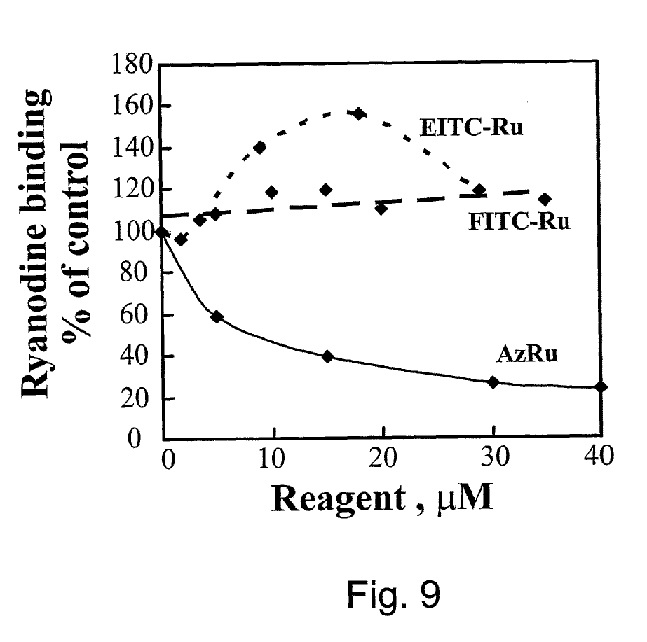

It was found that AzRu but not FITC-Ru or EITC-Ru inhibits the calcium activated ryanodine binding by SR (FIG. 9), with C₅₀ being about 10 μM.

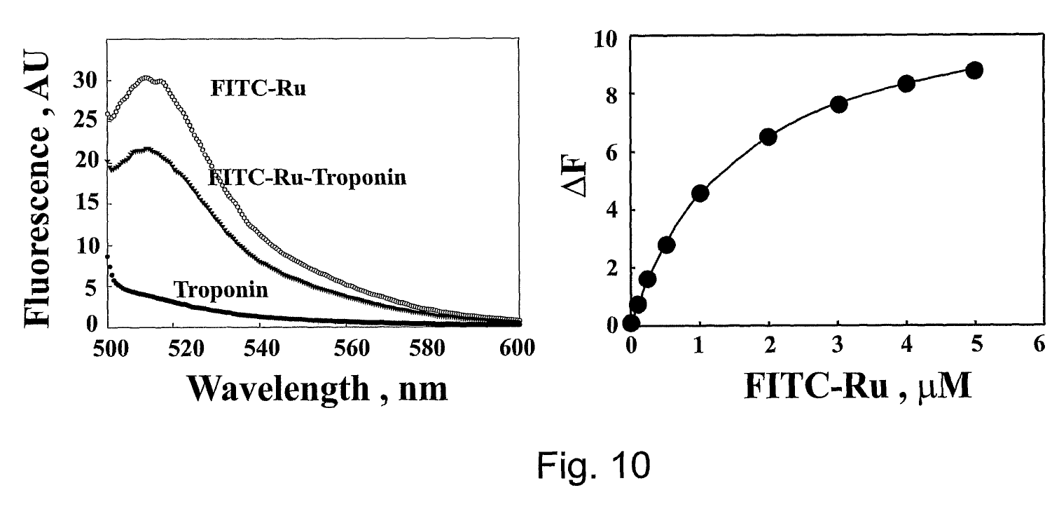

FITC-Ru interaction with purified troponin resulted in fluorescence quenching (FIG. 10A). The difference in the fluorescence intensity (ΔF) between free and protein-bound FITC-Ru was used to follow FITC-Ru binding kinetics (FIG. 10B), providing C₅₀ of 1 μM.

Following the above results, other fluorescent Ru-based compounds were prepared, analogously by reacting AzRu with fluorescent reagents. The reagents being selected from rhodamine isothiocyanate (RITC), tetramethyl rhodamine isothiocyanate (TRITC), and cyanine Cy5, thereby obtaining different fluorescent Ru-based (FlRu) reagents. For the sake of brevity, the fluorescent agent prepared by reacting an activated fluorophore X with AzRu are abbreviated as X-Ru herein; e.g., the compound produced from FITC and AzRu is abbreviated as FITC-Ru. It is appreciated that the abbreviation does not imply which parts of the original activated fluorophore remain in the new structure on which the agent is based.

In view of the above mentioned diversity of CaBP in the signal pathways, in their structures may be expected, and naturally each protein may interact differently with a low-molecular weight reagent. On the other hand, different moieties bound to AzRu derivative will interact differently with a certain protein. And here, without committing ourselves to any theory of reagent-protein interactions, comes into action another aspect of the instant invention—a factor of fluorescent moiety, the factor affecting not only the mode of detection, due to, e.g., different wavelength maxima, but affecting also the mode of interaction, due to different chemical group involved in the FlRu reagent.

It is known that there are several practical problems met in fluorescent measurements, which may complicate obtaining good results, and eventually decrease the contrast, or lead to fading, etc. Further, the cells have auto-fluorescence that may interfere with reagent fluorescence. Moreover, in cellular environment there may be materials which may otherwise interfere with the fluorescent signal. Of course, different fluorescent reagents will be affected differently, and it is, therefore, another advantage of the invention that it provides a plurality of different fluorophores, differing in excitation and emission wavelengths, enabling to select an optimal FlRu reagent for a given experiment. In another aspect, the possibility to select the fluorescent moiety from a plurality of structures will enable to distinguish between different CaBP, the differences between two fluorophores influencing not only the fluorescent signal, but also the chemical interaction of the small molecule with CaBP.

The invention, therefore, provides a fluorescent probe with a multi-mode action. In a first aspect—the fluorescent Ru-reagent interacts with a calcium-binding protein and visualizes the binding by fluorescence changes; in a second aspect—the radioactive Ru-reagent interacts with a calcium-binding protein and visualizes the binding site by radioactivity; in a third aspect—at least two FlRu reagents differing in the fluorophore part interact with a group of calcium-binding proteins and distinguish between at least two types of CaBP; in a fourth aspect—a reagent is selected from a plurality of FlRu to conform best to the experimental conditions.

In an important aspect of the invention, the fluorescence emitted by the fluorescent ligand is sensitive to small changes in environment, being enhanced or quenched or its wavelength being shifted, which is used for real-time monitoring of the interaction between FlRu and the CaBP. In a preferred embodiment, therefore, association and dissociation rates, binding affinities, and competition with other substrates are determined; and specificities are characterized.

In a preferred embodiment, the FlRu reagents are used in living cells to track the distribution of proteins that bind a certain FlRu reagent under certain conditions, to detect the temporal and spatial changes of said proteins. Lateral dynamics of the proteins using continuous time imaging [Dahan M. et al.: Science 302 (2003) 442-5] is preferably analyzed. Changes in cell fluorescence images, as monitored by fluorescence or confocal microscopy are used to follow the distribution and redistribution of FlRu-labeled CaBP in living cells, both under normal and signaling conditions (induction of apoptosis or muscle cell contraction). In case the auto-fluorescence of the biological sample overlaps with the reagent fluorescence, advantageously a reagent with non-overlapping emission can be selected.

A skilled person will appreciate that the new fluorescent probe of the invention provides a new approach to monitoring dynamics of Ca²⁺-binding proteins, offering also an opportunity to clarify whether redistribution of such proteins occurs upon various signaling, such as during apoptosis, muscle contraction, and other. The results obtained by the instant inventors suggest that the FITC-Ru probe may have no effect on cell survival, but may rather protect against the cell death, thus enabling to visualize the Ca²⁺-binding proteins redistribution in a non-destructive manner, and opening the possibility to characterize translocation of Ca²⁺-binding proteins between different compartments, such as the nucleus, ER, mitochondria, and plasma membrane.

FlRu agents according to this invention can be synthetized from radiolabeled reagents, thus enabling labeling of calcium binding proteins not only by a fluorescent tag, but also by radioactive tag, preferably comprising ¹⁰³Ru. Autoradiography, e.g., then helps to localize suspected proteins. Such double labeling enables discovering new protein links in the signal chains, explaining still unclear aspects of regulations and signal transduction pathways depending on calcium ions, separating and characterizing the involved peptides and proteins, as well as determining their structure and function.

The results show that FlRu, such as FITC-Ru and EITC-Ru, inhibit the activity of proteins which mediate Ca²⁺ transport such as the muscle Ca²⁺-pump, the mitochondrial Ca²⁺ uniporter and the voltage-dependent channel protein (VDAC). The compounds according to this invention also inhibit the activity of proteins possessing regulatory Ca²⁺-binding sites, such as the activity of calmudulin-activated proteins. It has been further found, on the other hand, that FITC-Ru has no effect on Ca²⁺ independent proteins such as hexokinase, alkaline phosphatase and glucose-6-phosphate dehydrogenase.

Based on the above findings, the present invention is also directed to a composition for use in diagnostic methods comprising identifying disorders associated with modified quantities of certain CaBP or by modified ratio of two types of CaBP in certain cells. Preferably, the diagnostic use relates to characterizing or visualizing CaBP associated with pathological states to be diagnosed, wherein the fluorescent agents of the invention, FlRu, are used for reactions in vitro.

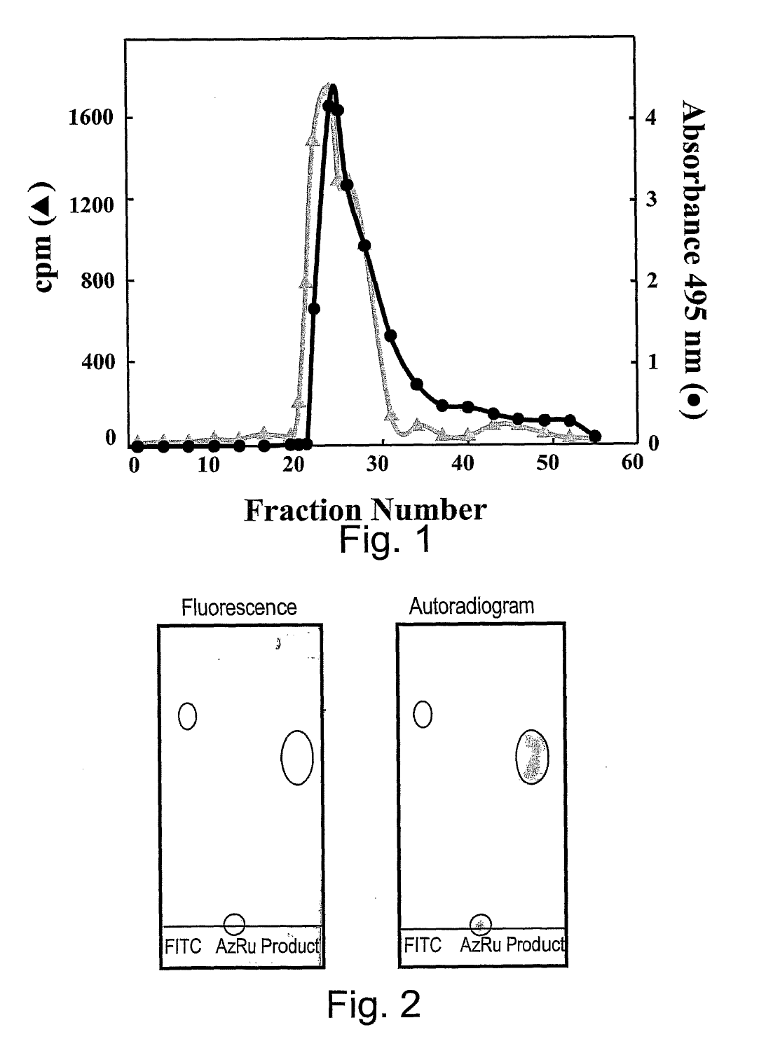

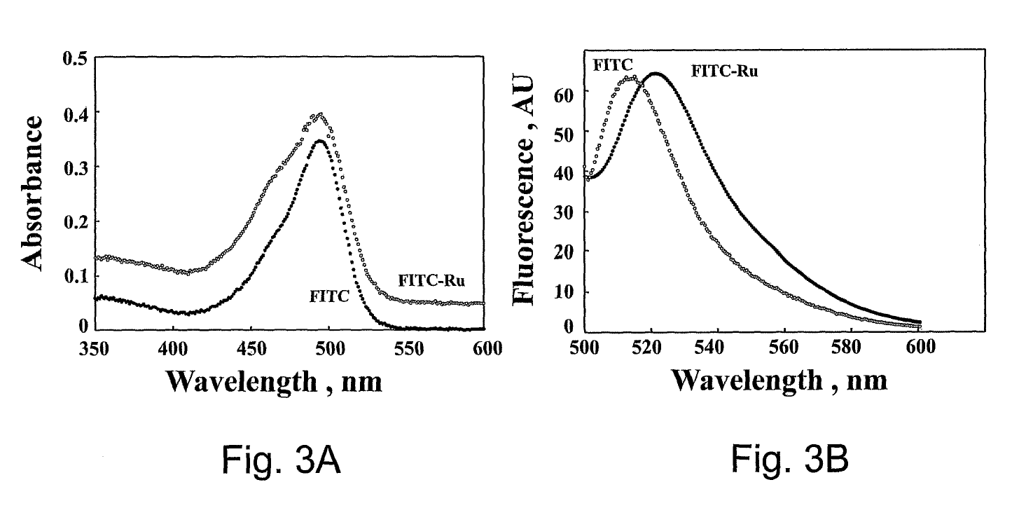

The invention provides a process for preparing ruthenium-based fluorescent compounds, binding to Ca²⁺-binding proteins, comprising the steps of: i) providing AzRu reagent, ii) reacting said AzRu with an activated fluorescent compound, and iii) purifying the product from the reactants. Said step i) may comprise preparing said reagent AzRu according to WO 2005/079155. Said activated fluorescent compound may be selected, for example, from FITC, EITC, RITC, TRITC, and Cy5. Said step may comprise chromatography, wherein the fractions with the product may be identified by measuring absorbance, fluorescence, radioactivity, or other properties associated with the product. The process of this invention provides a fluorescent agent, binding to CaBP, by reacting, e.g., FITC with AzRu, wherein the agent migrates as a single spot with Rf being about 0.6, when characterized by TLC with silica gel F₂₅₄ plates, using a solvent composed of 1M ammonium formate 80% and methanol 20%, pH 8.5 (FIG. 2), said product having an absorbance maximum at about 495 nm and a maximal fluorescence at 515 nm (FIG. 3), similarly as FITC. A radioactive FITC-A¹⁰³Ru was synthetized and purified (FIGS. 1 and 2).

The invention, thus, enables to characterize Ca²⁺-binding proteins, their intracellular and intra-organellar distribution, their binding sites and their possible stimuli-induced sub-cellular translocation. The invention, in some aspects, aims at characterizing the interaction of the fluorescent FlRu reagents with Ca²⁺-binding proteins, monitoring inhibition of their activities and fluorescence intensity changes upon the interaction. In an important aspect, the invention aims at developing plurality of FlRu probes for binding to CaBP, tracking the movement of said CaBP in living cells, and monitoring their spatial pattern and temporal dynamics. Fluorescence imaging, using fluorescence microscopy, may be monitored, wherein cells are subjected to different signaling. In order to identify the sub-cellular localizations of the proteins, interacting with the novel reagents, specific antibodies and confocal microscopy may be used, as well as electron microscopy when gold particles labeled secondary antibodies are used. It is understood that elucidation of new elements in the complex signaling pathways needs multidisciplinary attitudes, and requires plurality of reagents; here, the invention will contribute immensely. The invention will be optimally utilized when employed with other known techniques, including the photoreactive agent AzRu, and other agents.

The invention will be further described and illustrated in the following examples.

### Examples

### Materials and General Methods

Chemicals

ATP, CM-cellulose, lactate dehydrogenase, glutamate dehydrogenase, alkaline phosphatase, NAD+, NADH, EGTA, EDTA, Tris, MOPS, NaN₃ and RuCl₃ were obtained from Sigma. Ruthenium red (98% pure) was from Fluka. Sephadex LH-20 was obtained from Amersham Biosciences.

Preparations

AzRu was prepared as described in WO 2005/079155.

Sarcoplasmic reticulum (SR) membranes were prepared from rabbit fast twitch skeletal muscle as described by Saito [Saito et al.: J. Cell Biol. 99 (1984) 875-85]. Mitochondria were isolated from rat liver as described by us previously [Gincel D. et al.: Biochem J. 358 (2001) 147-55].

Assays

Protein concentrations were determined by the standard Lowry procedure [Lowry O. H. et al: J. Biol. Chem 193 (1951) 224-265]

### Example 1

FITC-ARu Synthesis

All steps were carried out in the dark. 10 mM FITC is contacted with 10 mM AzRu in 50% DMF, incubated overnight at room temperature. The sample was then applied to a Sephadex LH-20 column (2.5 cm×51 cm) pre-equilibrated with Tricine 10 mM, pH 7.4. The product was eluted with Tricine 10 mM, pH 7.4 (FIG. 1), while unreacted FITC was eluted with methanol.

The absorbance at 495 nm was measured and the peak was collected, lyophilized and analyzed by TLC on silica gel plates using ammonium formate 1M 80% and methanol 20%, pH=8.5 as a developer. The product migrated as a single spot with Rf=0.6 (FIG. 2). The absorbance (FIG. 3A) and emission spectra (3B) are shown, in which FITC (1 μM) and FITC-Ru (3 μM) were diluted in a 10 mM Tricine, pH 7.5. The emission spectrum was obtained with excitation at 495 nm. The FITC-Ru product had emission maximum at about 520 nm, whereas free FITC had maximum at about 515 nm.

The product is soluble in DMF and DMSO, less soluble in methanol, and insoluble in water. The infrared spectrum of the product indicates the absence of a specific peak representing a bound azido group. The structural formula of FITC-Ru was predicted from the LC/MS/MS analysis (FIG. 5).

### Example 2

FITC-Ru Inhibiting Ca²⁺ Accumulation in SR Membranes

SR was incubated for 10 min at 24° C. with the indicated concentrations of FITC-Ru. Ca²⁺ accumulation by SR was inhibited by FITC-Ru with half-maximal inhibition occurring at about 15 μM and 80%-90% inhibition occurring at 50 μM of FITC-Ru (FIG. 4).

### Example 3

FITC-Ru Inhibiting Ca²⁺-Accumulation in Mitochondria

Ca²⁺-accumulation in mitochondria was assayed for 1 min after 10 min incubation with the indicated concentration of FITC-Ru. Ca²⁺ accumulation by mitochondria was inhibited by FITC-Ru with half-maximal inhibition occurring at about 15 μM of FITC-Ru (FIG. 4).

### Example 4

FITC-Ru and AzRu Inhibition of the Ca²⁺-Accumulation in SR Membranes

SR was incubated for 10 min at 24° C. with the indicated concentrations of FITC-Ru or EITC-Ru. Ca²⁺ accumulation by SR was inhibited by EITC-Ru FITC-Ru, and AzRu with IC₅₀ occurring at about 7, 20 and 30 μM, respectively (FIG. 7).

### Example 5

EITC-Ru Inhibition of the Ca²⁺-Accumulation in Isolated Mitochondria

Ca²⁺ accumulation in mitochondria was assayed for 60 sec after 10 min incubation with the indicated concentration of EITC-Ru. Ca²⁺ accumulation by mitochondria was inhibited by EITC-Ru with half-maximal inhibition occurring at about 15 μM of FITC-Ru (FIG. 8).

### Example 6

AzRu but not FITC-Ru and EITC-Ru inhibits ryanodine-binding by SR membranes. For the determination of [³H]ryanodine binding, SR membranes were incubated for 20-60 min at 37° C. in a standard binding solution containing 1M NaCl, 20 mM MOPS (pH 7.4), 50 μM free Ca²⁺ and 20 nM [³H]ryanodine. Unbound ryanodine was separated from protein-bound ryanodine by vacuum filtration of the sample through nitrocellulose filters (0.45 μm), followed by two washes with 4 ml ice-cold buffer containing 0.2 M NaCl, 10 mM MOPS (pH 7.4) and 50 μM CaCl₂. The retained radioactivity in the dried filters was determined by liquid scintillation counting. Specific binding of [³H]ryanodine was defined as the difference between the binding in the presence of 20 nM [³H]ryanodine and in the presence of 20 μM unlabeled ryanodine. While AzRu inhibited ryanodine binding to SR membranes, no inhibition, but rather stimulation, of ryanodine-binding in SR, was obtained by FITC-Ru and EITC-Ru (FIG. 9).

### Example 7

FITC-Ru Binding to Troponin

The emission spectra of purified troponin, or FITC-Ru or troponin bound FITC-Ru were assayed at 24° C. using a PerkinElmer LS 55 fluorimeter. The excitation used was 495 nm, arbitrary units (AU) are shown in FIG. 10.

While this invention has been described in terms of some specific examples, modifications and variations are possible. It is therefore understood that within the scope of the appended claims, the invention may be realized otherwise than as specifically described.

1-40. (canceled)

41. A fluorescent ruthenium-containing compound (FlRu) which binds to a calcium-binding protein (CaBP), wherein said FlRu binds to troponin with affinity characterized by an association constant of at least about 10⁵.

42. The compound of claim 41 comprising a fluorescent moiety selected from the group consisting of fluorescein, eosin, rhodamine, and cyanine.

43. The compound of claim 42, wherein said moiety and ruthenium are in a molar ratio 1:1.

44. The compound of claim 41, comprising isotope ¹⁰³Ru.

45. A method of characterizing a calcium-binding protein (CaBP) in a source containing CaBP, comprising
i) providing a source containing a CaBP; and
ii) contacting said source with a FlRu compound according to claim 1, whereby said CaBP is bound to said compound.

46. A method according to claim 45, wherein said FlRu binds non-covalently to said CaBP, thereby labeling the CaPB by fluorescent tag.

47. A method according to claim 45, wherein said FlRu specifically binds to said calcium-binding protein, thereby inhibiting its calcium-binding activity.

48. A method according to claim 45, wherein said calcium-binding protein is selected from the group consisting of proteins involved in signal transduction, muscle contraction, neurotransmitter release, hormone secretion, cell motility, apoptosis, fertilization, cell proliferation, cell mitosis and in gene expression; proteins associated with Ca²⁺-transport, Ca²⁺-pumps, and with the mitochondrial uniporter; channel protein VDAC; Ca²⁺-release channel/ryanodine receptor; IP₃ receptor proteins involved in Ca²⁺-efflux in mitochondria; and soluble Ca²⁺ binding proteins regulating various cellular activities.

49. A method according to claim 45 comprising detecting a calcium-binding protein (CaBP) in a source containing CaBP, comprising
i) providing a source containing a CaBP;
ii) contacting said source with a FlRu compound of claim 1, whereby said CaBP is bound to said compound; and
iii) removing unbound FlRu;
thereby identifying CaBP as a fluorescent material that remains after the removal of unbound low-molecular FlRu in step iii).

50. A method according to claim 45, wherein said source comprises an item selected from the group consisting of a living cell, an in vitro sample, an array of microsamples, and a protein chip.

51. A method according to claim 45 comprising isolating a calcium-binding protein from a source comprising the same, which method comprises the steps of:
i) providing a source containing a CaBP;
ii) contacting said source with a FlRu compound of claim 1, whereby said CaBP is bound to said compound;
iii) removing unbound FlRu; and
iv) isolating fluorescent material that remains after the removal of unbound low-molecular FlRu in step iii).

52. A method according to claim 45 comprising screening for calcium-binding proteins, comprising the steps of:
i) providing a test sample comprising proteins;
contacting said sample with a FlRu compound of claim 1 under conditions which allow noncovalent binding of said compound to calcium-binding proteins to form fluorescent protein complexes;
iii) isolating from said sample said fluorescent complexes; and optionally
iv) subjecting said complexes to conditions supporting the release of said compound from said complexes, thereby obtaining free calcium-binding proteins.

53. A method according to claim 45, comprising diagnosing a disorder associated with a defect in the function of a CaBP in a subject, comprising:
i) providing a source of a CaBP of said subject, and a control source of a CaBP of a normal subject;
ii) contacting said sources with a FlRu compound according to claim 1 under conditions suitable for binding to occur; and
iii) detecting the fluorescence in the sources.

54. The method of claim 53, comprising:
i) optionally processing said sources to enrich them with said CaBP;
ii) contacting said sources with said compound under conditions suitable for binding to occur;
iii) detecting the fluorescence patterns in the samples; and
iv) comparing the pattern obtained in iii) for said subject with the pattern obtained for said control sample.

55. The method of claim 53, wherein said sources comprise cells or enriched protein samples.

56. The method of claim 53, wherein said sources comprise protein chips.

57. A process for preparing a fluorescent ruthenium-containing compound according to claim 1, comprising:
i) providing AzRu;
ii) contacting AzRu with a fluorescent reagent in a solvent;
iii) removing unreacted AzRu and unreacted activated compound, thereby obtaining said FlRu compound; and optionally
iv) removing solvents from said compound obtained in step iii).

58. The process of claim 57, wherein said AzRu and said fluorescent reagent are contacted at room temperature in a solvent selected from DMSO, DMF, or a mixture thereof with water.

59. The process of claim 57, wherein said fluorescent reagent is selected from FITC, EITC, RITC, TITC, and Cy5.

60. The process of claim 57, wherein said FlRu is a complex obtained by reacting FITC with AzRu, which complex has Rf of about 0.6 at TLC on cellulose F plates, using a developer comprising 80% of 1M ammonium formate and 20% of methanol.
